(19) United States
(12) Patent Application Publication
(10) Pub. No.: US 2007/0297951 A1
(43) Pub. Date: Dec. 27, 2007

(54) Apparatus for Reducing the Bacterial Content of the Air

(76) Inventor: Canio Caramuta, Salerno (IT)

Correspondence Address: THE WEBB LAW FIRM, P.C., 700 KOPPERS BUILDING, 436 SEVENTH AVENUE, PITTSBURGH, PA, 15219, US

(21) Appl. No.: 11/667,611

(22) PCT Filed: Jul. 27, 2005
(86) PCT No.: PCT/IT05/00446

§ 371 Date: May 11, 2007

(30) Foreign Application Priority Data
Nov. 12, 2004 (IT) FI2004A000237

(51) Int. Cl. A61L 9/20

(52) U.S. Cl. 422/121

(57) ABSTRACT

Apparatus for reducing the bacterial content of the air, of the type comprising at least a radiation chamber, provided with at least an ultraviolet radiation source and disposed between an entry for the air to be treated and an exit for the treated air, ventilation means to move the air between said entry and said exit, and means for shielding the ultraviolet radiation to prevent its exit by the same apparatus; apparatus wherein said shielding means include an optic labyrinth formed by walls developing, at least partially, according to a curve defining a path to prevent the passage of the ultraviolet radiation, allowing the free passage of the air.

The present invention concerns an apparatus for reducing the bacterial content of the air.

In particular, the invention relates to an apparatus for the sterilization of the air by means of ultraviolet radiations.

The technique which foresees the utilization of ultraviolet radiations acting on an air flow inside a chamber realized so as to prevent the exit of the same radiations is well known to obtain the sterilization of the air. In the apparatus which use such technique, the movement of the air is obtained by fans.

Apparatus for the sterilization of the air by ultraviolet radiations are described in EP-461310, GB-2215234, U.S. Pat. No. 5,894,130 and EP-1208853.

Among the inconveniences of the known apparatus for the sterilization by ultraviolet radiations there is their substantial complexity which negatively influences the production and the maintenance costs.

Another inconvenience is due to obstacles along the path followed by the flow of the air to prevent the exit of the ultraviolet radiations; these obstacles determine a high drag and pressure lowering, turbulences and aerodynamic noisiness. This makes necessary the utilization of fans featuring high prevalence, which are relatively noisy.

A further inconveniences of the known apparatus concern their prevalently vertical shape, the need of placing them to half height on a wall to make the complete re-circle of the treated air easier, and their encumbrance which makes relatively difficult the installation thereof in small spaces or relatively full of apparatus as in hospital environments. In other forms of realization of such an apparatus is not possible the fixing to a wall but exclusively at ceiling with consequent necessities of important wall modification, need of making false ceilings, etc.

Among the purposes of the present invention there is that to provide an apparatus able to eliminate the above-mentioned inconveniences.

This result has been achieved according to the invention thanks to the idea of producing an apparatus having the features described in the independent claims. Other features relate to the dependant claims.

Among the advantages of the present invention there it is that the system adopted to confine the radiations inside the apparatus itself foresees the substantial absence of obstacles along the path followed by the air and therefore it allows the use of fans with relatively limited prevalence and considerably more silent; that the present apparatus presents a remarkable constructive simplicity which determines less costs of production (i.e. costs for components and assembly) and of maintenance; that the disposition of the fan inside the radiation chamber contributes to keep the same fan sterile: in fact, the unavoidable stores of dust on the fan blades are continuously submitted to the germicidal radiation and cannot in such a way become contamination sources; that the noisiness is further reduced because of the loud attenuation determined by the walls of the radiation chamber; that the present apparatus presents relatively contained dimensions when compared with conventional apparatus and, therefore, it results easier to be positioned, also in small environments; that the path followed by the air flow, in particular thanks to the exit section dimensions, determines an outgoing jet which ensures a correct air mixing, avoiding the re-aspiration of the purified air; that the apparatus keeps its characteristics unchanged for its constructive simplicity even after a prolonged use.

These and other advantages and characteristics of the invention will be best understood by anyone skilled in the art from a reading of the following description in conjunction with the attached drawings given as a practical exemplification of the invention, but not to be considered in a limitative sense, wherein:

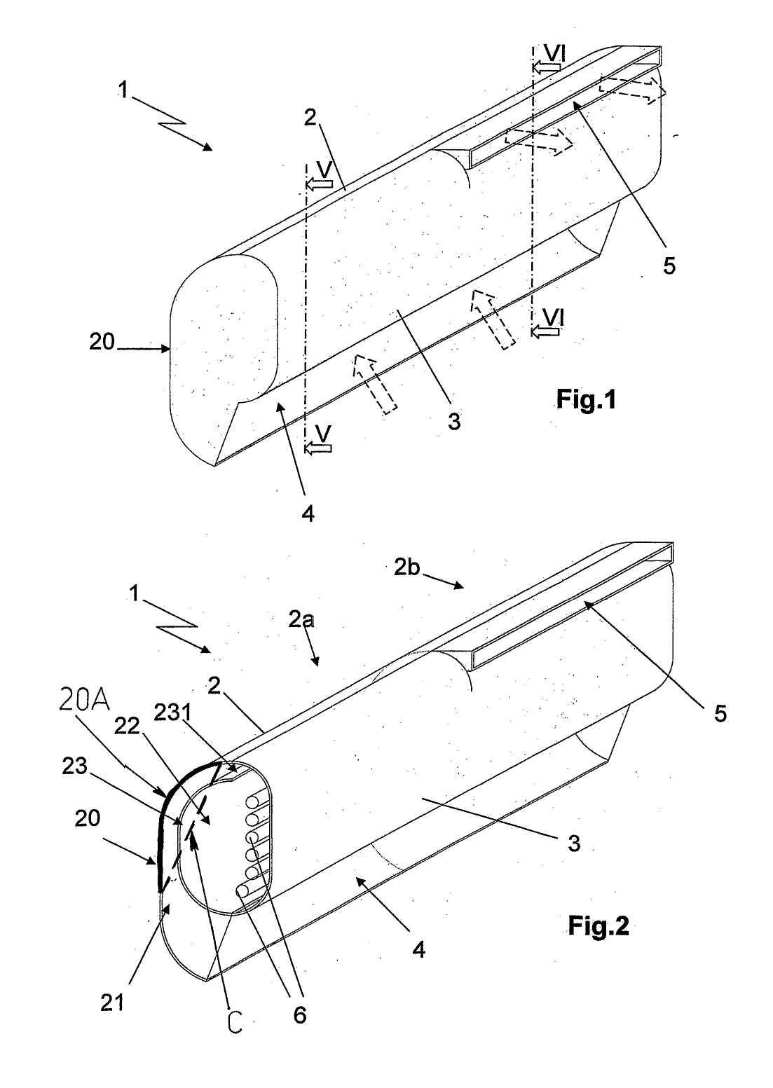

FIG. 1 is a schematic perspective view of a possible embodiment of an apparatus according the present invention;

FIG. 2 represents the embodiment of FIG. l in perspective view with removed parts for making better visible other parts;

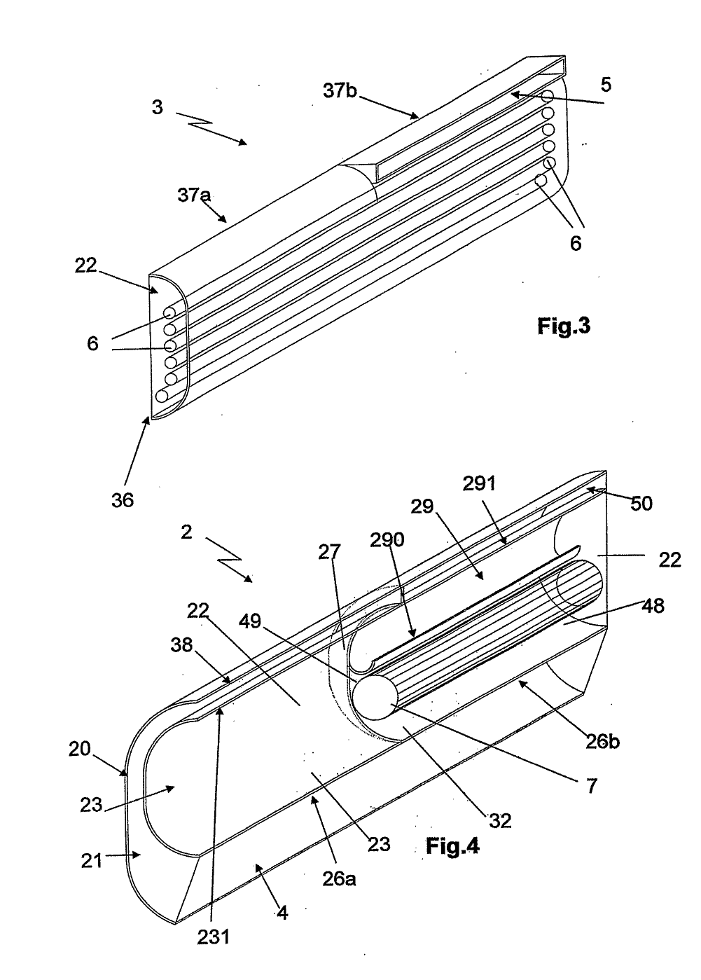

FIG. 3 is a schematic perspective view of a cartridge-cap of the example of FIG. l;

FIG. 4 is a schematic perspective view of a base of the example of FIG. 1;

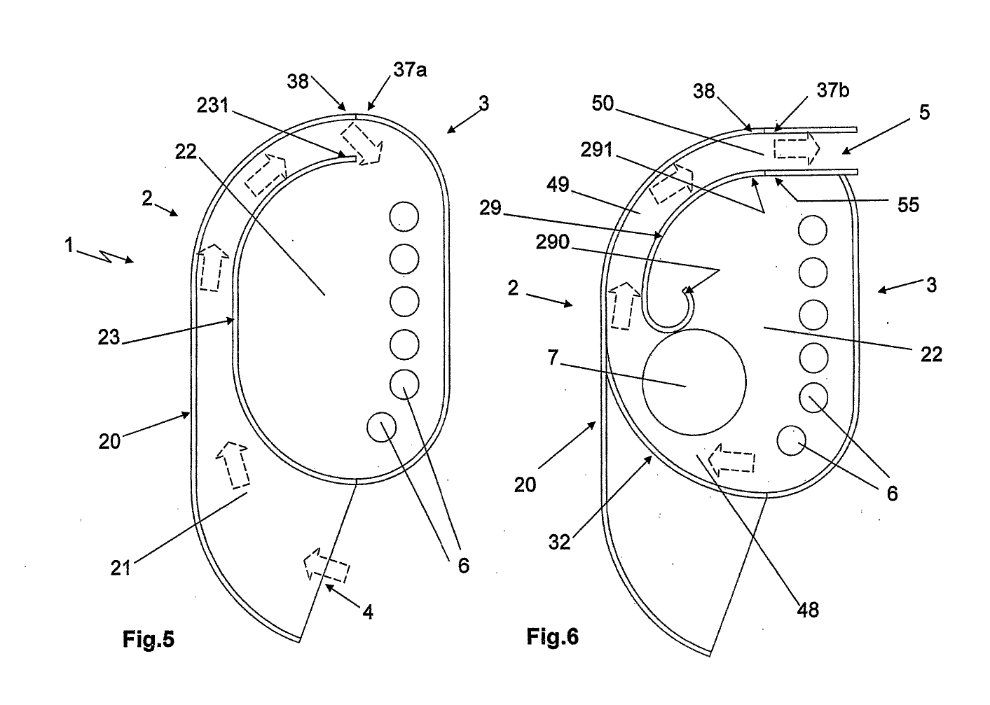

FIGS. 5 and 6 are two schematic view in cross section of the above example, according to the lines V-V and VI-VI of FIG. 1;

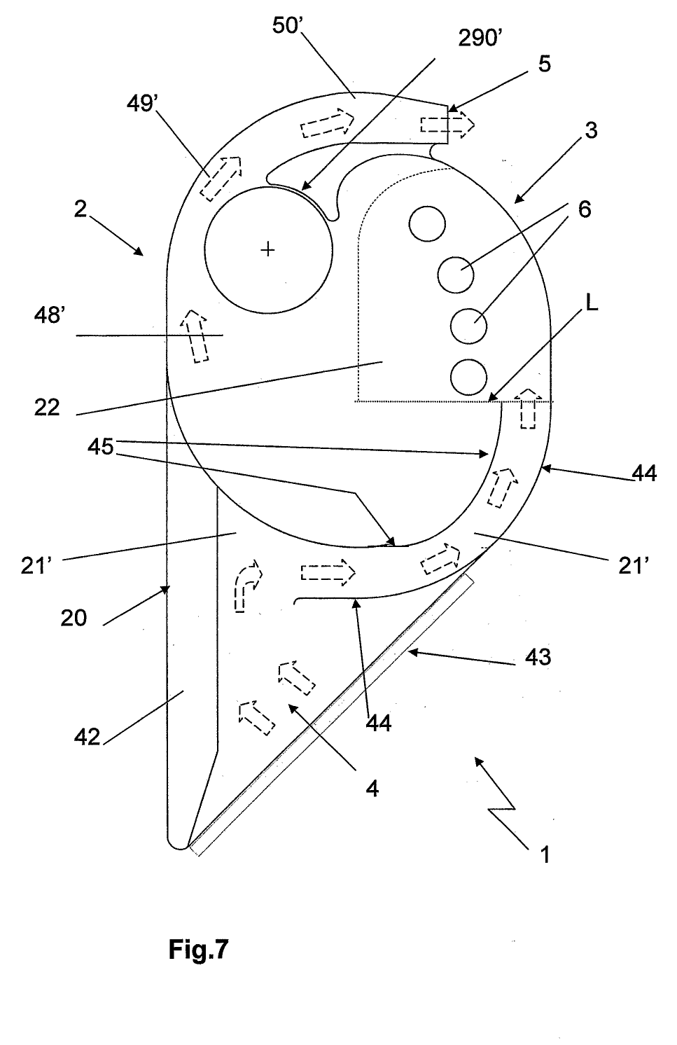

FIG. 7 is a schematic view in cross section of another embodiment of the present invention.

In the enclosed drawings an apparatus realized according to the invention was marked with the reference 1in his totality.

The apparatus 1, which is usable for reducing the bacterial content of the air, is of the type comprising at least a radiation chamber 22, provided with at least a ultraviolet radiation source 6, and disposed between an entry 4 for the air to be treated and an exit 5 for the treated air; the apparatus 1 is provided with ventilation means apt to move the air between said entry 4 and said exit 5 and with means for shielding the ultraviolet radiation in order to prevent its exit from the same apparatus. According to the invention said shielding means include an optic labyrinth formed by walls developing, at least partially, according to a curvilinear profile defining a path apt to prevent the passage of the ultraviolet radiation, allowing the free passage of the air.

Said optic labyrinth comprises an internal wall and an external wall. Both said external and internal walls have a curvilinear profile and thus delimit a curvilinear channel through which the flowing of air is allowed but ultraviolet radiations are intercepted, avoiding the exit thereof throughout the exit section 5.

In practice, as it will be clearer in the following, the walls which define the so-called optic labyrinth develop so that the innewall arranged internally to the curve intercepts the cord which is subtended to the correspondent arc of the externally disposed curve. For instance, referring to FIG. 2, the internal curve 23 is realized so that a cord C subtended to an arc 20A, represented with a thicker line, presented by the outside wall (in this part of the labyrinth the wall marked with 20), is intercepted by the internal curved wall 23. This prevents the free passage of the UV radiations and, at the same time, it does not obstruct in any way the free flow of the air. To increase the shielding effect, the channel walls are coated with a photo-absorbing material or, more simply, covered with opaque black paint. The cord C is not a physical element of the apparatus, but it is herein cited only for sake of clarity.

The apparatus 1 includes a base 2 and a cartridge-cap 3, associable to each other by screw means or any suitable joint, not shown, so as to define, as better afterwards described, the said radiation chamber 22.

A possible cartridge-cap 3 is shown as an example in FIG. 3. The cartridge-cap is constituted by a lid closing the base 2, provided with an opening which defines the exit 5 and with means apt to the emit ultraviolet radiations. In particular, according to this example, the cartridge-cap 3 is provided with six UV-C type tubes; the number of tubes may obviously be different. The internal side of the cartridge-cap 3 is coated with a material highly reflecting the UV radiations to increase the efficiency of the apparatus, for instance optic aluminium. The electric connectors which allow the connection of the tubes 6 to the supply are not represented. Also the part concerning the electronics of control and the supply circuit, which can be contained in the base 2, have been not represented to simplify the drawing.

A possible embodiment of the base 2 is represented in FIG. 4. The base 2 presents a rear face 20 which can be fixed to the wall of the environment in which sterilization of the air is to be executed, or to a different support structure such as a mobile pedestal.

The base 2 is divided, along its longitudinal development, into two sections 2a, 2b by a separator septum 27. In correspondence of a first section 2a (on the left in FIG. 4), the external wall 20 and a first internal wall 23, curvilinear shaped, delimitate an entry duct 21, which is upstream connected to the entry 4 of the apparatus and downstream to the radiation chamber 22. In practice, the entrance of the duct 21 coincides with the entry 4.

The wall 20, together with the correspondent wall of the cartridge-cap 3, defines a substantially spiral conformed profile.

In the second section 2b (to the right in the drawing) ventilation means 7 are provided, which are constituted by a tangential fan. In particular, the fan 7 (which can be also of axial or centrifugal type) is inside the radiation chamber 22; in this way it remains dipped in the flow of UV radiations and, therefore, it will also be kept sterile when unavoidable dust stores will form.

In the second section 2b an exit duct 48, 49, 50 is provided, which is connected upstream to the chamber 22 and downstream to the exit 5. Such a duct is delimited by the said outside wall 20, by a second internal wall 29, curvilinear profile-shaped, and by a third connecting wall 32, which lowerly is connected to the cartridge-cap 3. The profile defined by exit duct is substantially conformed spirally.

In particular, the lower edge 26b of the third connecting wall 32 constitutes the prolongation of the lower edge 26a of the first internal wall 23. In use configuration, i.e. with mounted apparatus, the edges 26a and 26b are in contact, airtight, with the lower edge 36 of the cartridge-cap 3. Analogously, the upper edge 38 of the base 2 is in contact, airtight, with the corresponding upper edge 37a, 37b of the cartridge-cap 3. The upper portion of the cap or lid 3 is differently conformed in correspondence of the two sections 2a and 2b of the base 2. In fact, in correspondence of the first section 2a, the edge 37a develops along a single line, while, in correspondence of the section 2b, it develops along two lines parallel respectively defining the real upper edge 37b and a second border 55, with which the upper border 291 of the second internal wall 29 of the base is in contact for delimiting the final portion 50 of the exit duct.

Besides, the said internal wall 29 has its lower extremity 290 more bent than the remaining part of the same wall 29 and is turned upwardly to constitute a so-called “stabilizing wall” of the whirl for the tangential fan 7, placed in closeness of the itself one, lowerly to the extremity 290, whose principal axis is parallel to the axis of longitudinal development of the wall 29. The said extremity 290 can also have other shape, allowing to define a stabilizing wall, obviously when this is suitable for the good working of the type of used fan. As regards the working, the air to be treated (whose entrance and exit flows in the FIGS. 1, 5 and 6 are schematically represented with dashed arrows), coming through the entry 4, enter the apparatus 1 in correspondence of the section 2a of the base 2, since the separator septum 27 prevents the air flowing towards the section 2b. The air flow therefore moves between the internal face of the wall 20 and the first internal wall 23 (walls which define the said duct entry 21) and, through the window defined by the edges 231 of the wall 23 and 38 of the wall 20, it reaches the radiation chamber 22, where it is submitted to the action of the radiating tubes 6.

Subsequently, the air flow horizontally moves towards the section 2b while it is subjected to the radiation of the UV-C lamps 6, it passes below the fan 7, in the zone of the duct of exit marked with 48 and therefore, passes in the zone 49 delimited by the internal face of the wall 20 and by the wall 29. The exit duct ends in correspondence of the exit 5, where it is marked with 50.

The entry duct 21 and the exit duct 48, 49, 50 thus define an optic labyrinth which is suitably shielded, for instance with a photo-absorbing opaque black coating.

The shape substantially spirally presented by the entry and exit ducts turns out especially favourable since also preventing the exit of the UV radiations noisiness allows an easy flow of the air, flow which can be realized also with a fan of reduced power and/or noisiness.

In FIG. 7 another possible embodiment of the invention, represented in cross section is visible. In this example, the apparatus presents a constant section, i.e. not interested by the subdivision between the right and left section, as in the previous example. Besides, it presents a longitudinal development (in width between right and left) relatively contained thanks to the particular conformation and disposition of the ducts for the air which allow a path which takes place without the longitudinal (horizontal) movement of the previous example. The particular compactness of this solution makes it advantageously usable not only in surgical rooms.

Always with reference to the FIG. 7, the base 2 and the cartridge-cap 3 are conformed differently with respect to the previous example, as inferable from the dashed line L which shows the subdivision.

Also in this case with 4 and 5 have been marked the entry for the air to be treated and, respectively, the exit of the treated air; the dashed arrows show the air flow.

Downstream the entry 4, after crossing a filter 43 which is extended for all the width of the entry 4, the air enters the equipment 1 through an entry duct 21′.

The duct 21′ is lowerly and upperly delimited by respective bent walls 44 and 45. In particular, the lower wall 44 is extended, for about a fourth of circumference, up to define the front face of the apparatus, while the upper wall 45 is at a fixed distance from the wall 44 so as to provide the duct 21′ with a hole having a substantially constant shape. The duct 21′ ends in the radiation chamber 22, in which four UV tubes 6 are provided. Also in this example a tangential fan 7 has been used; fans of other type can also be used. In this case, the so-called stabilizing wall is defined by a wall 290′ of the base 2. The air flow exiting from the chamber 22 moves towards the exit 5, passing through the exit duct, in this case marked by the numerical references 48′, 49′ and 50′.

In FIG. 7, besides, with 42 a chamber destined to the electronics control has been marked.

Clearly, changes may be made to the form, dimensions, component part locations, and type of materials employed in the embodiment described and illustrated herein without, however, departing from the scope of the present invention.

1-10. (canceled)

11. An apparatus for reducing the bacterial content ofthe air comprising:
a radiation chamber, provided with at least an ultraviolet radiation source and disposed between an entry for the air to be treated and an exit for the treated air;
ventilation means for moving the air between said entry and said exit; and
means for shielding the ultraviolet radiation for preventing exit of the ultraviolet radiation by the same apparatus;
wherein said means for shielding includes an optic labyrinth formed by walls developing, at least partially, according to a curve defining a path to prevent the passage of the ultraviolet radiation therefrom, and allowing the free passage of the air.

12. The apparatus of claim 11, wherein the walls which define said curve comprise an internal wall and an external wall, the internal wall arranged to correspond to the curve intercepting a cord which is subtended to a correspondent arc of the external wall.

13. The apparatus of claim 11, wherein said optic labyrinth has a substantially spiral shaped profile.

14. The apparatus of claim 11, wherein said ventilation means comprise a fan disposed inside said radiation chamber.

15. The apparatus of claim 14, wherein said fan is a tangential fan disposed inside said radiation chamber, in a zone in communication with the relevant radiations of the UV-source.

16. The apparatus of claim 14, wherein said ventilation means further comprise a stabilizing wall for the fan.

17. The apparatus of claim 11, further comprising:
a base and a cartridge-cap which, when associated, define said radiation chamber;
said base is divided, along its longitudinal development, into a first section and a second section by a separator septum; and
said first section of said base comprising an external wall and an internal wall having a curvilinear shape, delimitating an air entry duct, which is positioned upstream of said entry and downstream of said radiation chamber, and defining, with a corresponding wall of said cartridge-cap, a substantially spiral shaped profile.

18. The apparatus of claim 17, wherein:
said ventilation means are disposed in said second section of said base, in which an air exit duct, connected upstream to said chamber and downstream to said exit, is delimitated by said external wall, by a second internal wall which is curvilinear profile-shaped, and by a third connecting wall, which is at least partially connected to the cartridge-cap; the profile defined by said exit duct being substantially spirally shaped.

19. The apparatus of claim 18, wherein:
said second internal wall comprises an upwardly turned lower extremity having a greater bend than at least a portion of the second internal wall; and
a tangential fan is provided downwardly to said extremity, having a principal axis substantially parallel to an axis of longitudinal development of said second internal wall.

20. An apparatus for reducing the bacterial content of the air, comprising:
a radiation chamber, provided with an ultraviolet radiation source, and disposed between an entry for the air to be treated and an exit for the treated air;
ventilation means for moving the air between said entry and said exit; and
means for shielding the ultraviolet radiation to prevent exit of the ultraviolet radiation.

21. The apparatus of claim 20, wherein said ventilation means further comprise a fan disposed within said radiation chamber, in a zone in communication with the relevant radiations of the UV-source.
